(19) United States
(12) Patent Application Publication
(10) Pub. No.: US 2023/0389794 A1
(43) Pub. Date: Dec. 7, 2023

(54) BINOCULAR VIDEO STEREO OPHTHALMOSCOPE

(71) Applicant: Baylor College of Medicine, Houston, TX (US)

(72) Inventor: Omar Solyman, Houston, TX (US)

(21) Appl. No.: 18/029,188

(22) PCT Filed: Sep. 27, 2021
(86) PCT No.: PCT/US2021/071604

§ 371 Date: Mar. 29, 2023

Related U.S. Application Data
(60) Provisional application No. 63/087,050, filed on Oct. 2, 2020.

(51) Int. Cl. A61B 3/13; A61B 3/14; H04N 13/344

(52) U.S. Cl. CPC A61B 3/132; A61B 3/145; H04N 13/344

(57) ABSTRACT

A binocular optical device for a retinal examination such as a binocular video stereo ophthalmoscope. The binocular video stereo ophthalmoscope may include two synchronized camera modules: a first camera having a first field of view, the first camera configured such that the first field of view is in a line-of-sight of a wearer of the binocular optical device; and a second camera having a second field of view, wherein the second camera is configured such that the second field of view at least partially overlaps the first field of view. Additionally, the optical device may include a memory, and a processor coupled to memory so that the processor may implement a method for performing improved eye examinations. For example, the processor may be configured to record, into the memory, first video data from the first camera synchronized with second video data from the second camera.

### CROSS-REFERENCE TO RELATED PATENT APPLICATIONS

This application claims the benefit of priority of U.S. Provisional Patent Application No. 63/087,050 filed Oct. 2, 2020 and entitled “Binocular Video Stereo Ophthalmoscope,” which is hereby incorporated by reference.

### FIELD OF THE DISCLOSURE

The instant disclosure relates to optical tools. More specifically, portions of this disclosure relate to optical tools for performing eye examinations.

### BACKGROUND

The back of the eye includes a retina, which is a transparent film that captures light rays and converts them to electrical signals for processing by the brain, an optic disc, which is the tip of the optic nerve that conducts the vision signal to the brain, and the choroid, which underlies and nourishes the retina. Examination of the back of the eye is a practice used by ophthalmologists in diagnosing diseases of the eyes and other diseases that may be present in the patient. Examining the back of the eye can reveal sight-threating conditions like age-related macular degeneration, diabetic retinopathy, glaucoma, and other diseases. Additionally, examination of the eye can provide information regarding life-threatening diseases like brain tumors manifested as optic disc swelling, retina tumors (e.g., retinoblastoma), and choroidal tumors (such as melanoma and metastasis). A conventional examination of the eye involves an indirect ophthalmoscope in a procedure referred to as ophthalmoscopy. A conventional tool for this procedure is a set of prisms and mirrors that converge the sight from each of the examiner's eyes to a narrow beam that can pass through the pupil of the patient's eye, with an additional condensing lens focusing an image at the back of the eye between the patient and examiner.

### SUMMARY

A binocular optical device for retinal examination, such as a binocular video stereo ophthalmoscope, may be used to examine the back of a patient's eyes. The binocular video stereo ophthalmoscope may be implemented according to different embodiments of the disclosure in order to allow simultaneous examination and recording of the same view of the patient's eyes, allow spare time required to examine the patient again for photographic documentation, improve the results of a fundus examination in less cooperative patients, record data as a stereoscopic view for illustrating three dimensions, and/or providing an erect non-inverted image of the back of the patient's eyes (approximately 180 degrees rotation of the image instead of an inverted image of the posterior segment of the eye), and/or allow flexible examination locations by providing improved portability of the examination apparatus. Such a binocular optical device in some embodiments may improve upon difficulties with examination using conventional tools, such as not having a video record of the examination to improve record keeping and/or allow supervision. Further, the image quality of the back of the patient's eye examined with a tool according to some embodiments of this disclosure may be higher than that seen by the examiner through some conventional tools.

According to some embodiments, a binocular optical device for retinal examination may include a first camera having a first field of view, the first camera configured such that the first field of view is in a line-of-sight of a wearer of the binocular optical device; a second camera having a second field of view that at least partially overlaps the first field of view; a memory; and a processor coupled to memory, the first camera, and the second camera, wherein the processor is configured to record, into the memory, a first video data from the first camera synchronized with a second video data from the second camera.

In some embodiments, the processor may be configured to process the first video data and the second video data to generate a stereoscopic video data. In some embodiments, the processor may be further configured to process the first video data and the second video data to generate an anatomically corrected view. In some embodiments, the processor may be further configured to generate the anatomically corrected view without using mirrors by processing the first video and the second video by vertically inverting and laterally reversing the first video data and the second video data through video data processing techniques performed within the processor or a second processor (such as a graphics processing unit GPU) coupled to the processor.

In certain embodiments, the apparatus may further include a virtual reality (VR) headset including a first display configured to display a left image and a second display configured to display a right image, wherein the first display and the second display are coupled to the processor and may be integrated in the VR headset, and wherein the processor is configured to display the anatomically corrected view to a user as a three-dimensional view using the first display and the second display of the headset. In some embodiments, the processor is configured to display the anatomically corrected view in real-time as first and second video data is received from corresponding first and second cameras during an examination.

In another embodiment, the apparatus may further include a first viewing lens and a second viewing lens, wherein the first camera is oriented behind the first viewing lens, wherein the second camera is oriented behind the second viewing lens, and/or wherein the first viewing lens and the second viewing lens are part of a binocular indirect ophthalmoscope.

According to some embodiments, the apparatus may further include a coaxial illumination source configured to illuminate an illuminated scene, wherein the first camera and the second camera are configured such that the first field of view and the second field of view are at least partially overlapping with the illuminated scene. In some embodiments, the first camera and the second camera may be mounted to the coaxial illumination source, and the first camera and the second camera may be separated by an inter-cameral distance of less than approximately three millimeters.

According to some embodiments, a method of processing video data for an eye examination may include receiving a first video data from a first camera of a binocular video stereo ophthalmoscope during a retinal examination, wherein the first video data corresponds to a first field of view, and the first field of view is in a line-of-sight of a wearer of the binocular video stereo ophthalmoscope; receiving a second video data from a second camera during the retinal examination, wherein the second video data corresponds to a second field of view that at least partially overlaps with the first field of view; and recording the first video data synchronized with the second video data to a memory during the retinal examination. The method may be performed during operation of an apparatus, such as the embodiments of an apparatus disclosed herein. In some embodiments, the method is performed by a processor or a combination of hardware and/or software components of an apparatus of an embodiment disclosed herein.

In some embodiment, the method may further include generating a stereoscopic video data based on the first video data and the second video data. In some embodiments, the method may further include generating an anatomically corrected view based on the first video data and the second video data; and switching between the anatomically corrected view and a non-anatomically corrected view based on user input. In certain embodiments, the anatomically corrected view may be generated without using mirrors by processing the first video and the second video by vertically inverting and laterally reversing the first video data and the second video data.

According to other embodiments, the method may further include displaying a left image on a first display of a headset and a right image on a second display of the headset, wherein the headset is configured to display the anatomically corrected view to a user in three-dimensions using the first display and the second display of the headset. In another embodiment, the anatomically corrected view may be displayed on the headset in real-time. In a different embodiment, the method may further include illuminating an illuminated scene, wherein the first video data of the first camera and the second video data of the second camera are at least partially overlapping with the illuminated scene.

The method may be embedded in a computer-readable medium as computer program code including instructions that cause a processor to perform the steps of the method. In some embodiments, the processor may be part of an information handling system.

As used herein, the term “coupled” means connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially parallel includes parallel), as understood by a person of ordinary skill in the art.

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

Further, an apparatus or a system that is configured in a certain way is configured in at least that way, but it can also be configured in other ways than those specifically described.

The terms “comprise” (and any form of comprise, such as “comprises” and “comprising”), “have” (and any form of have, such as “has” and “having”), and “include” (and any form of include, such as “includes” and “including”) are open-ended linking verbs. As a result, an apparatus or system that “comprises,” “has,” or “includes” one or more elements possesses those one or more elements, but is not limited to possessing only those elements. Likewise, a method that “comprises,” “has,” or “includes,” one or more steps possesses those one or more steps, but is not limited to possessing only those one or more steps.

The foregoing has outlined rather broadly certain features and technical advantages of embodiments of the present invention in order that the detailed description that follows may be better understood. Additional features and advantages will be described hereinafter that form the subject of the claims of the invention. It should be appreciated by those having ordinary skill in the art that the conception and specific embodiment disclosed may be readily utilized as a basis for modifying or designing other structures for carrying out the same or similar purposes. It should also be realized by those having ordinary skill in the art that such equivalent constructions do not depart from the spirit and scope of the invention as set forth in the appended claims. Additional features will be better understood from the following description when considered in connection with the accompanying figures. It is to be expressly understood, however, that each of the figures is provided for the purpose of illustration and description only and is not intended to limit the present invention.

### BRIEF DESCRIPTION OF THE DRAWINGS

For a more complete understanding of the disclosed system and methods, reference is now made to the following descriptions taken in conjunction with the accompanying drawings.

FIG. 1 is a schematic block diagram illustrating a binocular optical device for retinal examination according to some of the embodiments of the disclosure.

FIG. 2 is a flow chart illustrating a method for processing video data for a retinal examination according to some embodiments of the disclosure.

FIG. 3 is an illustration of a binocular optical device for retinal examination according to some embodiments of the disclosure.

FIG. 4 is a schematic block diagram illustrating an apparatus according to some embodiments of the disclosure.

### DETAILED DESCRIPTION

A new tool for examining the back of a patient's eyes described in embodiments herein may be referred to as a binocular video stereo ophthalmoscope. The following example embodiments describe and illustrate various features and descriptions of how the invention is embodied in an apparatus and/or integrated into an information handling system and how it is an improvement of methods and processes used in a medical application. It should be understood, however, that the detailed description and the specific examples, while indicated as embodiments of the disclosure, are given by way of illustration only, and not by way of limitation. Various substitutions, modifications, additions, and/or rearrangements within the spirit and/or scope of the underlying inventive concept will become apparent to those having ordinary skill in the art from this disclosure.

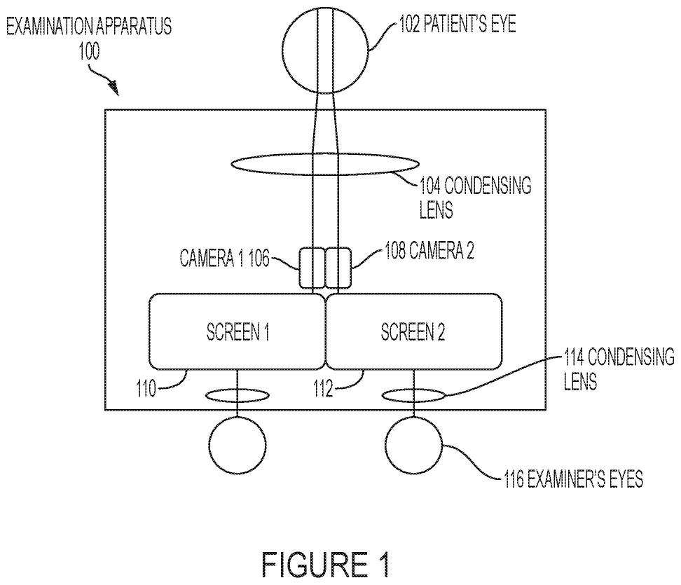

An illustration of the ophthalmoscope according to one embodiment of the disclosure is shown in FIG. 1. An examination apparatus 100, such as a binocular video stereo ophthalmoscope according to different embodiments of the disclosure, may allow simultaneous examination and recording of the same view of the patient's eyes 102. For example, the examination apparatus 100 may include two camera modules, camera 1 106 and camera 2 108. In some embodiments, the cameras may be arranged such that camera 1 106 having a first field of view is in a line-of-sight of a wearer of the binocular optical device 100 and arranged such that camera 2 108 has a second field of view that at least partially overlaps the first field of view. The video data from the cameras 106 and 108 may be synchronized such that a three-dimensional image may be recreated using the video data from cameras 106 and 108. The examiner's eyes 114 may view the camera images on a screen 1 110 and/or a screen 2 112. With the help of a convex lens such as condensing lens 104 in front of camera 1 106 and camera 2 108 and a convex lens 114 of appropriate power in front of each of the examiner's eyes, the simultaneous binocular stereoscopic fundus examination and identical recording of the same view of the patient's eyes are feasible.

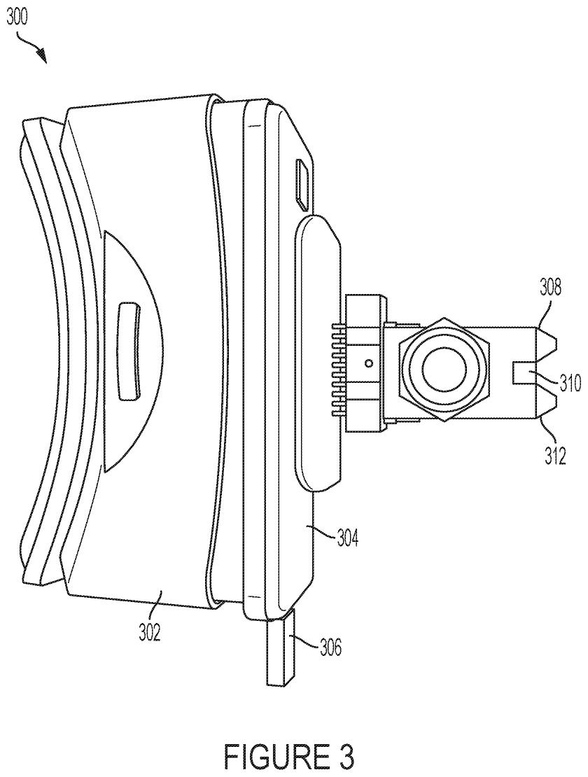

Additionally, the examination apparatus 100 may allow spare time required to examine the patient again for photographic documentation, improve the results of a fundus examination in less cooperative patients, record data in stereoscopic three dimensions, provide an erect non-inverted image of the back of the patient's eyes (approximately 180 degrees rotation of the image instead of an inverted image of the posterior segment of the eye), and/or allow flexible examination locations by providing improved portability of the examination apparatus. In some embodiments, the ophthalmoscope may allow switching between the anatomically corrected view described herein and an inverted image as generated by conventional techniques based on user input to the processing apparatus, such as through a dedicated button on the headset as shown in FIG. 3 or through a user interface (UI) display.

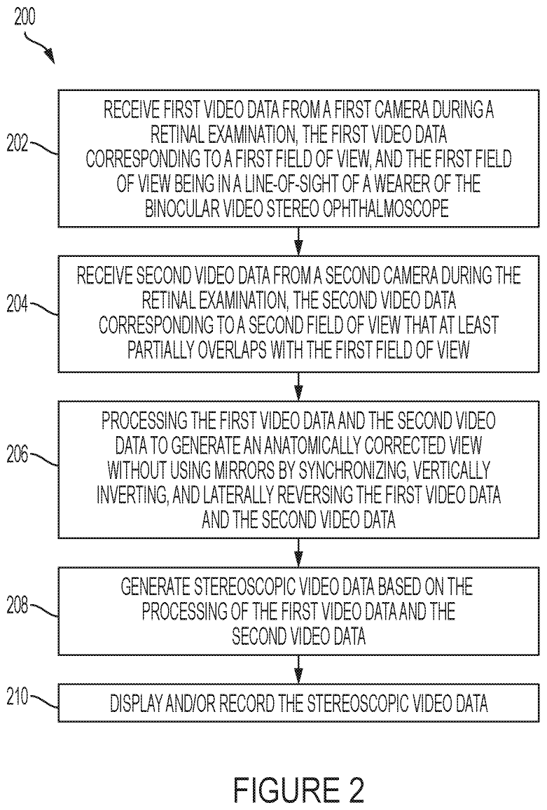

One method of operation of a binocular video stereo ophthalmoscope according to some embodiments of the disclosure is shown in FIG. 2 that illustrates method 200. At block 202, an apparatus, such as a binocular video stereo ophthalmoscope, may receive first video data from a first camera during a retinal examination, and the first video data may correspond to a first field of view that may be in a line-of-sight of a wearer of the apparatus.

The apparatus may receive second video data from a second camera at block 204 during the retinal examination. The second video data may correspond to a second field of view that at least partially overlaps with the first field of view. After block 204, the apparatus may process the first video data and the second video data to generate an anatomically corrected view without using mirrors by synchronizing, vertically inverting, and laterally reversing the first video data and the second video data. The apparatus may then generate a stereoscopic video data based on the processing of the first video data and the second video data at block 208. For the examiner, the apparatus may then display and/or record the stereoscopic video data at block 210.

This schematic flow chart diagram is generally set forth as a logical flow chart diagram. Likewise, other operations for the circuitry are described without flow charts herein as sequences of ordered steps. In some embodiments, the steps are performed in real-time to allow an examiner to examine the back of the eye by providing instructions to the patient and observing the resulting change in view of the back of the eye corresponding to the patient following the examiner's instructions. A view may be real-time, despite delays in signaling or processing of the video data that may add less than, for example, one second, or less than half a second, or less than 100 milliseconds. A view may also be real-time, despite delays in transmission of the first video data and the second data and/or the anatomically corrected view across a network, such as the Internet, to a remote examiner. The depicted order, labeled steps, and described operations are indicative of aspects of methods of the invention.

Other steps and methods may be conceived that are equivalent in function, logic, or effect to one or more steps, or portions thereof, of the illustrated method. For example, the processing of the first video data and the second video data to generate the anatomically-corrected view may be performed after recording the first video data and second video data as a raw data stream that may be processed off-line at a later time. Additionally, the format and symbols employed are provided to explain the logical steps of the method and are understood not to limit the scope of the method. Although various arrow types and line types may be employed in the flow chart diagram, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

FIG. 3 illustrates an apparatus 300 that may implement the method 200. For example, apparatus may include a virtual reality (VR) headset 300 that may be coupled to a computing device 304, having for example a processor and memory, for processing video data and sensor data received by the VR headset 300. In some embodiments, components of the computing device 304, such as a processor and memory, are incorporated in the VR headset 300. The examiner during the examination may wear two lenses, such as high-index of refraction lenses, that are part of the virtual reality (VR) headset 300. The lenses may be coupled to a first camera 1 308 and a second camera 312 that may be separated with a light source 310. For example, the binocular video stereo ophthalmoscope examination apparatus 300 may include a headset with two cameras placed a few millimeters apart from each other, such that the two cameras have an overlapping, but different, field of view. According to different embodiments, the binocular video stereo ophthalmoscope may include two synchronized camera modules: a first camera having a first field of view, the first camera configured such that the first field of view is in a line-of-sight of a wearer of the binocular optical device; and a second camera having a second field of view, wherein the second camera is configured such that the second field of view at least partially overlaps the first field of view.

In some embodiments, the examination apparatus 300 may include a light source 310, such as a coaxial illumination source, in the middle between camera 1 308 and camera 312. In certain embodiments, cameras, such as the camera 1 308 and\the camera 2 312, may be mounted to the coaxial illumination source. For example, the first camera and the second camera may be separated by an inter-cameral distance of less than approximately three millimeters.

Additionally, the VR headset 300 may include a memory such as a storage medium and/or an interface connection 306 for coupling to computing device 304. For example, the computing device 304 may include an interface connection 306 such as a Universal Serial Bus (USB) connection. In some embodiments, the smartphone or other processing apparatus may include memory for storing information from the examination for later review and/or verification of the examination. For example, a memory in the smartphone or processing apparatus may store the first video data and the second video data in a synchronized manner before or after the processing to generate the anatomically corrected view.

The foregoing has outlined rather broadly certain features and technical advantages of embodiments of the present invention in order that the detailed description that follows may be better understood. It should be appreciated by those having ordinary skill in the art that the conception and specific embodiment disclosed may be readily utilized as a basis for modifying or designing other structures for carrying out the same or similar purposes. For example, other displays, such as accessory teaching mirrors, televisions, or computer monitors, may be used to display the anatomically corrected view of the patient's eyes, rather than two screens in a virtual reality (VR) headset. It should also be realized by those having ordinary skill in the art that such equivalent constructions do not depart from the spirit and scope of the invention as set forth in the appended claims.

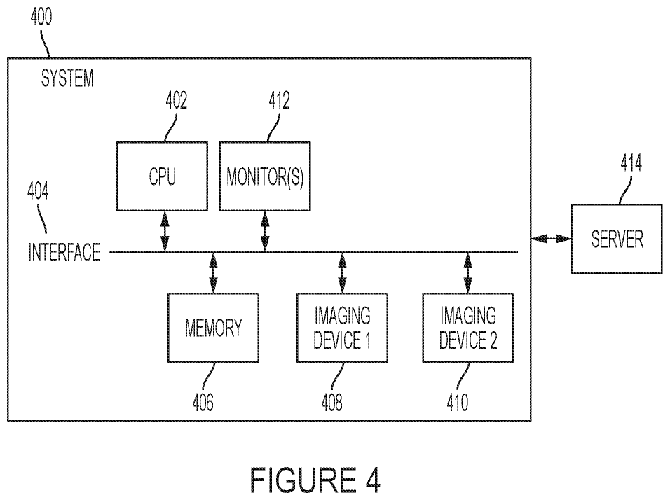

In other embodiments, the examining apparatus may include a system 400 shown in FIG. 4. System 400 may include a central processing unit (CPU) 402 that may be a general-purpose CPU, microprocessor, or the like. The CPU 402 may communicate through a wired connection or wireless connection via a bus 404. The bus 404 may be coupled to a memory 406. For example, the CPU 402 may access a Random Access Memory (RAM) 406 of the IHS 400.

The system 400 may further include an imaging device 1 408 and an imaging device 2 410. For some embodiments, imaging device 1 408 and/or imaging device 2 410 may include a camera module. For example, the camera module may be coupled to a smartphone or other processing apparatus (e.g., a device with a processor coupled to a memory, wherein the memory includes computer-readable instructions that configure the processor to process video data from the camera modules) for processing of images within the video data received from the first and second camera modules. In some embodiments, the camera modules may be wired to a smartphone or other computing device through Universal Serial Bus (USB) cables. As another example, the camera modules may be coupled to a communication device that transmits images through a Wi-Fi wireless network or other communications system to a smartphone or other computing device.

In certain embodiments, the system 400, such as a processing device receiving a first video data from the imaging device 1 408 (e.g., a first camera) and a second video data from the imaging device 2 410 (e.g., a second camera) may be configured to process the first video data and the second video data to generate a stereoscopic video data. In some embodiments, the processor 402 is further configured to process the first video data and the second video data to generate an anatomically corrected view. For example, the examiner has the option to anatomically correct the real-time examination view of the ocular fundus by performing a 180-degree rotation of each image separately. In some embodiments, the anatomically corrected view is generated without using mirrors by processing the first video and the second video by vertically inverting and laterally reversing the first video data and the second video data. Although the video data is described as processed by the CPU 402 or a separate computing device, the examination apparatus, such as VR headset 300, may include fixed-function hardware configured for performing the vertical inversion and lateral reversal of the video data from each imaging device 408 and 410.

Furthermore, the anatomically corrected view may be displayed on one or more monitor(s) 412. The IHS 400 may be a headset such as the headset 300 that may be configured to display the anatomically corrected view to a user as a three-dimensional view using the first display and the second display of the headset. In some embodiments, the processor or CPU 402 may be configured to display the anatomically corrected view in real-time. For example, the first display or monitor(s) 412 may be configured to display a left image and a second display or monitor(s) 412 may be configured to display a right image. In certain embodiments, a processed version of the first video data may be presented on the first screen while a synchronized, processed version of the second video data may be presented on the second screen. In some embodiments, the IHS 400, smartphone, or other processing apparatus may include memory for storing information from the examination for later review and/or verification of the examination. For example, a memory in the smartphone or processing apparatus may store the first video data and the second video data in a synchronized manner before or after the processing to generate the anatomically corrected view. In certain embodiments, the first video data, the second video data, or other data may be stored on a separate server 414 that may be connected to IHS 400 via a system bus or another interface.

Although the present disclosure and certain representative advantages have been described in detail, it should be understood that various changes, substitutions and alterations can be made herein without departing from the spirit and scope of the disclosure as defined by the appended claims. For example, the first video data, the second video data, and/or the anatomically-corrected view may be compressed using image compression techniques, such as described in video standards such as H.264 (AVC), H.265 (HEVC), VP9, and AV1, to facilitate transport of the video data through circuitry interconnects and communication networks. As another example, other processing may be applied to the video data, such as to enhance brightness or dynamic range of the image.

Any suitable processor-based device may be utilized including, without limitation, personal data assistants (PDAs), computer game consoles, and multi-processor servers. Moreover, the present embodiments may be implemented on application specific integrated circuits (ASIC) or very large scale integrated (VLSI) circuits. In fact, persons of ordinary skill in the art may utilize any number of suitable structures capable of executing logical operations according to the disclosed embodiments. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized. Accordingly, the following claims are intended to include within their scope such processes, machines, manufacture, compositions of matter, means, methods, or steps.

What is claimed is:

1. A binocular optical device for retinal examination, the binocular optical device comprising:
a first camera having a first field of view, the first camera configured such that the first field of view is in a line-of-sight of a wearer of the binocular optical device;
a second camera having a second field of view, wherein the second camera is configured such that the second field of view at least partially overlaps the first field of view;
a memory; and
a processor coupled to the memory, the first camera, and the second camera, wherein the processor is configured to record, into the memory, first video data from the first camera synchronized with second video data from the second camera.

2. The apparatus of claim 1, wherein the processor is further configured to process the first video data and the second video data to generate a stereoscopic video data.

3. The apparatus of claim 1, wherein the processor is further configured to process the first video data and the second video data to generate an anatomically corrected view.

4. The apparatus of claim 3, wherein the processor is further configured to generate the anatomically corrected view without using mirrors by processing the first video and the second video by vertically inverting and laterally reversing the first video data and the second video data.

5. The apparatus of claim 3, further comprising a virtual reality (VR) headset comprising a first display configured to display a left image and a second display configured to display a right image, wherein the headset is coupled to the processor, and wherein the processor is configured to display the anatomically corrected view to a user as a three-dimensional view using the first display and the second display of the headset, wherein the processor is configured to display the anatomically corrected view in real-time.

6. The apparatus of claim 1, further comprising a first viewing lens and a second viewing lens, wherein the first camera is oriented behind the first viewing lens, wherein the second camera is oriented behind the second viewing lens, and wherein the first viewing lens and the second viewing lens are part of a binocular indirect ophthalmoscope.

7. The apparatus of claim 1, further comprising a coaxial illumination source configured to illuminate an illuminated scene, wherein the first camera and the second camera are configured such that the first field of view and the second field of view are at least partially overlapping with the illuminated scene.

8. The apparatus of claim 7, wherein the first camera and the second camera are mounted to the coaxial illumination source, and wherein the first camera and the second camera are separated by an inter-cameral distance of less than approximately three millimeters.

9. The apparatus of claim 1, wherein the apparatus comprises a binocular video stereo ophthalmoscope.

10. A method, comprising:
receiving a first video data from a first camera of a binocular video stereo ophthalmoscope during a retinal examination, wherein the first video data corresponds to a first field of view, and the first field of view is in a line-of-sight of a wearer of the binocular video stereo ophthalmoscope;
receiving a second video data from a second camera during the retinal examination, wherein the second video data corresponds to a second field of view that at least partially overlaps with the first field of view; and
recording the first video data synchronized with the second video data to a memory during the retinal examination.

11. The method of claim 10, further comprising generating a stereoscopic video data based on the first video data and the second video data.

12. The method of claim 10, further comprising generating an anatomically corrected view based on the first video data and the second video data; and switching between the anatomically corrected view and a non-anatomically corrected view based on user input.

13. The method of claim 12, wherein the anatomically corrected view is generated without using mirrors by processing the first video and the second video by vertically inverting and laterally reversing the first video data and the second video data.

14. The method of claim 10, further comprising displaying a left image on a first display of a headset and a right image on a second display of the headset, wherein the headset is configured to display the anatomically corrected view to a user in three-dimensions using the first display and the second display of the headset.

15. The method of claim 14, wherein the anatomically corrected view is displayed on the headset in real-time.

16. The method of claim 10, further comprising illuminating an illuminated scene, wherein the first video data of the first camera and the second video data of the second camera are at least partially overlapping with the illuminated scene.

17. A computer program product comprising:
a non-transitory computer readable medium comprising instructions for causing an information handling system to perform the steps comprising:
receiving a first video data from a first camera of a binocular video stereo ophthalmoscope during a retinal examination, wherein the first video data corresponds to a first field of view, and the first field of view is in a line-of-sight of a wearer of the binocular video stereo ophthalmoscope;
receiving a second video data from a second camera during the retinal examination, wherein the second video data corresponds to a second field of view that at least partially overlaps with the first field of view; and
recording the first video data synchronized with the second video data to a memory during the retinal examination.

18. The computer program product of claim 17, wherein the non-transitory computer readable medium further comprises instructions for generating a stereoscopic video data based on the first video data and the second video data.

19. The computer program product of claim 17, wherein the non-transitory computer readable medium further comprises instructions for generating an anatomically corrected view based on the first video data and the second video data; and switching between the anatomically corrected view and a non-anatomically corrected view based on user input.

20. The computer program product of claim 17, wherein the non-transitory computer readable medium further comprises instructions for displaying a left image on a first display of a headset and a right image on a second display of the headset, wherein the headset is configured to display the anatomically corrected view to a user in three-dimensions using the first display and the second display of the headset.
